(19) United States
(12) Patent Application Publication
(10) Pub. No.: US 2020/0123227 A1
(43) Pub. Date: Apr. 23, 2020

(54) INTERFERON PRODRUG FOR THE TREATMENT OF CANCER

(71) Applicant: The Board of Regents of the University of Texas System, Austin, TX (US)

(72) Inventors: Yang-Xin FU, Dallas, TX (US); Xuezhi CAO, Dallas, TX (US)

(73) Assignee: The Board of Regents of the University of Texas System, Austin, TX (US)

(21) Appl. No.: 16/624,648

(22) PCT Filed: Jun. 18, 2018
(86) PCT No.: PCT/US2018/037982

§ 371 Date: Dec. 19, 2019

Related U.S. Application Data
(60) Provisional application No. 62/522,564, filed on Jun. 20, 2017.

(51) Int. Cl. C07K 14/715; C07K 14/56; C07K 14/565

(52) U.S. Cl. CPC C07K 14/7156; C07K 14/56; C07K 2317/52; C07K 2319/30; C07K 14/565

(57) ABSTRACT

The present disclosure is directed to interferon prodrugs and their use in treating cancer. A particular advantage to such constructs is their ability to exert powerful anti-tumor activity in vivo reduction in many of the significant toxicities associated with interferon therapy.

### PRIORITY CLAIM

This application claims benefit of priority to U.S. Provisional Application Ser. No. 62/522,564, filed Jun. 20, 2017, the entire contents of which are hereby incorporated by reference.

### BACKGROUND

### 1. Field

The present disclosure relates generally to the fields of medicine, oncology and immunotherapeutics. More particularly, it concerns the development and use of immunoreagents in the treatment of cancers.

### 2. Background

Type I interferons (IFNs) are thought to directly suppress tumor cell proliferation. They have been used with success for the treatment of several types of cancer, including hematological tumors (chronic myeloid leukemia, hairy cell leukemia, multiple myeloma, and Non-Hodgkin lymphomas), and solid tumors (melanoma, renal carcinoma, and Kaposi's sarcoma)(Ferrantini et al., 2007, Moschos & Kirkwood, 2007, Zitvogel et al., 2015, and Antonelli et al., 2015). Indeed, accumulating evidence shows that endogenous type I IFN plays a key role in educating DC for increasing cross priming against tumor antigens for enhancing the anti-tumor activity of chemotherapies, radiotherapies, targeted therapies and immunotherapies (Schiavoni et al., 2011, Burnette et al., 2011; Stagg et al., 2011 and Woo et al., 2014).

A particular advantage of type I IFN treatment is due to its ability to intervene at multiple points in the generation of anti-tumor immune responses, including stimulation of the innate and adaptive cytotoxic lymphocyte populations, negative regulation of suppressive cell types, its impact on tumor cells by inhibiting proliferation, and by modulating apoptosis, differentiation, migration and cell surface antigen expression (Parker et al., 2016). Importantly, some of these actions could represent a potential strategy for use of type I IFNs in overcoming the resistance of cancers to immunotherapy. One of the mechanisms for relapse in cancer patients is the lack of T cell recognition due to down-regulation of expression of MHC class I molecules and peptide transporter genes for tumor antigen presentation (Sharma et al., 2017). Type I IFNs can be used to induce MHC class I expression, as wekk as LMP2/7 and TAP-1/2 expression in tumor cells, together being ideal an combination strategy to combat therapeutic resistance to immunotherapy (Khanna, 1998). Additionally, type I IFNs can be used to suppress Tregs and MDSC by negatively regulating the proliferation of Tregs (Pace et al., 2010 and Srivastava et al., 2014) and reducing the accumulation and suppressive function of MDSCs (Zoglmeier et al., 2011), both of which directly suppress cytotoxic T lymphocyte activity (Schmidt et al., 2012 and Gabrilovich et al., 2009). These multiple anti-tumor effects make type I IFNs intriguing anti-cancer drugs, both in a monotherapies and in combination with other therapies.

However, one of the biggest barriers to the use of type I IFNs in the clinic is the severe side effects associated with such treatments. The most frequently encountered side effects are flu-like symptoms, hematological toxicity, elevated transaminases, nausea, fatigue, and psychiatric sequelae. These side effects hamper reaching and maintaining the doses needed for maximal therapeutic effect, and their occurrence can outweigh clinical benefit of type I IFN treatment entirely (Kreutzer et al., 2004, Sleijfer et al., 2005 and Lotrich, 2009). Therefore, the ability to specifically deliver type I IFNs to the tumor microenvironment is imperative for continued clinical use of type I IFN. Strategies are needed to modify type I IFN in order to obtain new forms of the drug which only exert their activity in and at the tumor, and also to achieve avoidance of severe adverse effects outside the tumor.

### SUMMARY

Thus, in accordance with the present disclosure, there is provided an interferon prodrug comprising (a) an interferon alpha and beta receptor (IFNAR) domain that retains IFN binding activity; (b) a type 1 interferon (IFN) domain that retains type 1 interferon activity when not engaged by said IFNAR domain; (c) an immunoglobulin (Ig) Fc domain, (d) a first linker fused at one end to the N-terminus of said IFN and fused at the other end to said IFNAR, wherein said first linker is protease cleavable; and (e) a second linker fused at one end to the C-terminus of said IFN and fused at the other end to the N-terminus of said Ig Fc domain. The Ig may be IgG, such as IgG1 or IgG2. The interferon prodrug may contain two copies of said type 1 IFN domain. The interferon prodrug may contain more than two copies of said type 1 IFN domain. The first linker may be cleavable by one or more matrix metalloproteinases, such as MMP1, MMP3, MMP9, MMP10, MMP11, MMP12, MMP13 or MMP14. The first linker may be cleavable by UPA, FAPa and/or Cathepsin B. The linker may be G4S-SUB1-G4S-SUB2-G4S-SUB3-G4S, wherein SUB1-3 are distinct enzyme cleavage sites. The IFNAR may be IFNAR1 or IFNAR2. The IFN may be IFN-α, IFN-62, IFN-κ, IFN-δ, IFN-ε, IFN-τ, IFN-ω, or IFN-ξ.

In another embodiment, there is provided a nucleic acid construct encoding an interferon prodrug comprising (a) an interferon alpha and beta receptor (IFNAR) domain that retains IFN binding activity; (b) a type 1 interferon (IFN) domain that retains type 1 interferon activity when not engaged by said IFNAR domain; (c) an immunoglobulin (Ig) Fc domain, (d) a first linker fused at one end to the N-terminus of said IFN and fused at the other end to said IFNAR, wherein said first linker is protease cleavable; (e) a second linker fused at one end to the C-terminus of said IFN and fused at the other end to the N-terminus of said Ig Fc domain; and (f) a promoter positioned 5′ to the 5′ end of said IFNα domain. The Ig may be IgG, such as IgG1 or IgG2. The interferon prodrug contains two copies of said type 1 IFN domain. The interferon prodrug may contain more than two copies of said type 1 IFN domain. The first linker may be cleavable by a matrix metalloproteinase, such as MMP1, MMP3, MMP9, MMP10, MMP11, MMP12, MMP13 and/or MMP14. The first linker may be cleavable by UPA, FAPa and/or Cathepsin B. The linker may be G4S-SUB1-G4S-SUB2-G4S-SUB3-G4S, wherein SUB1-3 are distinct enzyme cleavage sites. The IFNAR may be IFNAR1 or IFNAR2. The IFN may be IFN-α, IFN-62, IFN-κ, IFN-δ, IFN-ε, IFN-τ, IFN-ω, or IFN-ξ.

Also provided are a recombinant cell expressing an interferon prodrug as defined above; a recombinant cell comprising a nucleic acid construct as defined above; a method of expressing an interferon prodrug comprising culturing the cell defined above; a method of expressing an interferon prodrug comprising culturing the cell defined above, and the use of an interferon prodrug as defined above (a) in the preparation of a medicament for the treatment of cancer; or (b) for the treatment of a cancer.

In another embodiment, there is provided a method of treating cancer comprising administering to a subject in need thereof an interferon prodrug as defined above. The method may further comprise the step of assessing protease expression in a cancer cell obtained from said subject. The cancer cell may be obtained from a biopsy, or may be a circulating tumor cell. The cancer may be lung cancer, breast cancer, brain cancer, oral cancer, esophageal cancer, head & neck cancer, skin cancer, stomach cancer, liver cancer, pancreatic cancer, renal cancer, ovarian cancer, prostate cancer, bladder cancer, colon cancer, testicular cancer, uterine cancer, cervical cancer, lymphoma, or leukemia. The cancer may be primary, recurrent, metastatic or multi-drug resistant. The patient may have previously received surgical therapy, chemotherapy, radiotherapy, hormonal therapy or immunotherapy.

The method may further comprise treating said subject with a second cancer therapy, such as a surgical therapy, chemotherapy, radiotherapy, hormonal therapy or immunotherapy. The subject may be a human or a non-human mammal. The method may further comprising administering said interferon prodrug more than once, such as daily, every other day, weekly, every other week, or monthly. The prodrug may be administered systemically, or intratumorally, local to a tumor or regional to a tumor. Treating may comprise one or more of slowing tumor growth, halting tumor growth, reduction in tumor size or burden, increasing survival as compared to an untreated subject, inducing cancer remission, inducing tumor cell apoptosis, or inducing tumor necrosis.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1. Schematic structure of IFN-Prodrug. IFNAR-ECD (blue), substrate linker (red), IFN (yellow), tumor-specific enzyme (green), IgG Fc (gray). The left N-terminal arm represents the intact IFN-Prodrug form with the IFNAR-ECD fused and bound to IFN, whereas the right N terminal arm represents an activated IFN-Prodrug from which the IFNAR-ECD has dissociated.

FIGS. 2A-C. Activated form of IFN-Prodrug shows increased IFN activity rather than restore. (FIG. 2A) hIg, IFNa4-Fc, IFNAR1-based IFN-Prodrug and IFNAR2-based IFN-Prodrug were serial-diluted in 5-fold from 20 nM. (FIGS. 2B-C) IFN-Prodrug was diluted to 1 μM in Assay Buffer (50 mM Tris, 10 mM CaCl2, 150 mM NaCl, 0.05% Brij-35 (w/v), pH 7.5 (TCNB)). Add rmMMP-9 to a final concentration of 1 ng/μL and incubate at 37° C. for 6 hours. (FIG. 2B) IFNa4-Fc, R1-NSUB without or with rmMMP-9 treatment and R1-SUB (MMP-2/9 substrate) without or with rmMMP-9 treatment were serial-diluted in 5-fold from 20 nM. (FIG. 2C) IFNa4-Fc, R2-NSUB (MMP-2/9 substrate) without or with rmMMP-9 treatment and R2-SUB without or with rmMMP-9 treatment were serial-diluted in 5-fold from 20 nM. Diluted fusion protein solutions were added into the RAW-Lucia-ISG reporter cells to stimulated luciferase secretion. Conditioned supernatants were harvested at 24 h post-stimulation for luciferase assay. The error bars represent the means±s.e.m. of triplicates.

FIG. 3. IFNAR2-based IFN-Prodrug shows better anti-tumor effect than IFNAR1-based IFN-Prodrug. C57BL/6 mice (n=5 per group) were injected s.c. with 5×10⁵ B16 cells and treated intraperitoneally with 1 nmol of hIg, IFNa4-Fc, R1-SUB (MMP2/9 substrate) or R2-SUB (MMP2/9 substrate) on days 11, 15 and 21. Tumor growth is monitored twice a week and reported as the mean tumor size ±s.e.m. over time.

FIGS. 4A-D. Enzymes expression in mouse normal and tumor tissues. C57BL/6 mice (n=4) were injected s.c. with 1×10⁶ MC38 cells or 5×10⁵ B16 cells. Indicated normal tissues and tumors were harvested on day 11. Intracellular RNA was extracted for RT-qPCR assay to determine (FIG. 4A) uPA, (FIG. 4B) MMP-2, (FIG. 4C) MMP-9, and (FIG. 4D) MMP-14 mRNA abundance. The results are presented as the percentage to 18sr. The error bars represent the means±s.e.m. of triplicates.

FIGS. 5A-E. IFN-prodrug improves safety without compromising efficacy. C57BL/6 mice (n=3 per group) were injected s.c. with 5×10⁵ B16 cells and treated intraperitoneally with 1 mM of hIg, IFNa4-Fc, or R2-SUB (MMP-14 substrate) on days 10, 13 and 16. Mice were bled and serum were collected on day 17. (FIG. 5A) The concentration of inflammatory cytokines IL-6, TNF, MCP-1 and IFN-g in serum was measured by mouse inflammation cytometric bead array (CBA). (FIG. 5B) The activity of ALT in serum was measured by the Reflotron Plus® System. (FIG. 5C) Tumor growth and (FIG. 5D) body weight were monitored twice a week. (FIG. 5E) Survival curve due to toxicity. Mice with body weight lose over 30% were considered to be dead. The error bars represent the means±s.e.m.

FIGS. 6A-H. Human proteases expression level between tumor and adjacent normal tissues. The DiffExp module of TIMER (Tumor IMmune Estimation Resource) website online analysis of the comparison of gene expression levels for all samples from TCGA (The Cancer Genome Atlas). (FIG. 6A) MMP-1, (FIG. 6B) MMP-3, (FIG. 6C) MMP-9, (FIG. 6D) MMP-10, (FIG. 6E) MMP-11, (FIG. 6F) MMP-12, (FIG. 6G) MMP-13, and (FIG. 6H) MMP-14.

FIGS. 7A-C. Activation of human ProIFN in vitro. (FIG. 7A) hIFNa2-Fc, hIFNAR1-based ProIFN and hIFNAR2-based ProIFN were serial-diluted in 10-fold from 50 μM. IFN activity was measured by 293T-Dual™ hSTING-R232 reporter cells. (FIG. 7B) Human hIFNAR2-based ProIFN with (hProIFN-Sub) or without (hProIFN-Nsub) the substrate were diluted to 1 μM in assay buffer (50 mM Tris, 10 mM CaCl₂, 150 mM NaCl, 0.05% Brij-35 (w/v), pH 7.5 (TCNB)). Add rmMMP-9 to a final concentration of 1 ng/μL and incubate at 37° C. for 6 hours. IFN activity was measured by 293T-Dual™ hSTING-R232 reporter cells. (FIG. 7C) Human hIFNAR2-based hProIFN-Sub or hProIFN-Nsub were diluted to 1 μM in assay buffer (50 mM Tris, 10 mM CaCl₂), 150 mM NaCl, 0.05% Brij-35 (w/v), pH 7.5 (TCNB)). Add rmMMP-9 to a final concentration of 1 ng/μL and incubate at 37° C. for 0, 0.5, 2, or 6 hours. Digested samples were separated on a Stain Free™ Gel. The gel was imaged with a stain-free enabled imager. The error bars represent the means±s.e.m. of triplicates.

### DESCRIPTION OF ILLUSTRATIVE EMBODIMENTS

Prodrugs are pharmacologically inactive chemical derivatives of drugs that require a transformation within the body in order to become active. They are designed to overcome pharmaceutic- and/or pharmacokinetic-based problems associated with the parent drug that would otherwise limit the clinical usefulness of the drug (Stella et al., 1985). Recently, a protease-activated antibody (pro-antibody) targeting vascular cell adhesion molecule 1 (VCAM-1), a marker of atherosclerotic plaques, was constructed by tethering a binding site-masking peptide to the antibody via a matrix metalloprotease (MMP) susceptible linker. The activity of such disease-associated proteases can be exploited to site-specifically target antibody activity in vivo (Erster et al., 2012). In a cancer therapy example, a screen-identified peptide that can block binding of anti-EGFR to EGFR target was linked to anti-EGFR antibody. The resulting epidermal growth factor receptor (EGFR)-directed pro-antibody markedly improved safety with increased half-life in non-human primates, enabling it to be dosed safely at much higher levels than cetuximab (Desnoyers et al., 2013). However, such peptides often have weak affinity, resulting in incomplete blockade of the drug protein, as well as strong immunogenicity that prevents longer treatment. Therefore, the inventors designed a new strategy based on a natural receptor having proper affinity, and lacking immunogenicity ing host, to block drug engaging of its receptor in non-tumor tissues. Specifically, they focused on type I interferons due to their due to their important role in the anti-tumor immune response to many cancers⁸.

The inventors designed an IFN-Prodrug using immunoglobulin (IgG) constant regions and IFNAR (IFNAR1 or IFNAR2) extracellular domains as an IFN activity blocker fused at the N-terminus of the IFN. The IFNAR is connected with a specific linker that is selectively cleavable by a protease overexpressed in the tumor microenvironment. This masks the toxic activity of the IFN domain until it arrives at the tumor, while the Fc fragment fused on the C-terminal end of IFN improves its half-life in vivo. This IFN-Prodrug showed significantly reduced IFN activity prior to cleavage of the linker, but regained its activity after enzyme cleavage. The inventors also demonstrated the efficacy and enhanced safety of IFN-Prodrug in vivo in a mouse B16-OVA melanoma model.

Thus, the advantage of using this IFN-Prodrug as a therapeutic for cancer are: 1) low toxicity; 2) surprisingly higher IFN activity in the activated form than parent IFN-Fc; 3) ease of production and purification with high yield; 4) specific targeting of tumor tissues; 5) use of non-immunogenic blocking reagent; 6) and personalized IFN-Prodrug design according to differing tumor enzyme expression. Such an IFN-Prodrug can therefore improve the safety profile of interferons without compromising efficacy, and thus may enable the wider use of advanced interferon therapy formats, such as antibody-cytokine bispecific fusion proteins. These and other aspects of the disclosure are described in greater detail below.

### I. TYPE 1 INTERFERONS

A. Interferon Types

Human type I interferons (IFNs) are a large subgroup of interferon proteins that help regulate the activity of the immune system. Interferons bind to interferon receptors. All type I IFNs bind to a specific cell surface receptor complex known as the IFN-α receptor (IFNAR) that consists of IFNAR1 and IFNAR2 chains. Type I IFNs are found in all mammals, and homologous (similar) molecules have been found in birds, reptiles, amphibians and fish species. The mammalian types are designated IFN-α (alpha), IFN-β (beta), IFN-κ (kappa), IFN-δ (delta), IFN-ε (epsilon), IFN-τ (tau), IFN-ω (omega), and IFN-ξ (zeta, also known as limitin). The IFN-α proteins are produced by leukocytes. They are mainly involved in innate immune response against viral infection. The genes responsible for their synthesis come in 13 subtypes that are called IFNA1, IFNA2, IFNA4, IFNA5, IFNA6, IFNA7, IFNA8, IFNA10, IFNA13, IFNA14, IFNA16, IFNA17, IFNA21. These genes are found together in a cluster on chromosome 9.

IFN-α is also made synthetically as medication in hairy cell leukemia. The International Nonproprietary Name (INN) for the product is interferon alfa. The recombinant type is interferon alfacon-1. The pegylated types are pegylated interferon alfa-2a and pegylated interferon alfa-2b.

The IFN-β proteins are produced in large quantities by fibroblasts. They have antiviral activity that is involved mainly in innate immune response. Two types of IFN-β have been described, IFN-β1 (IFNB1) and IFN-β3 (IFNB3) (a gene designated IFN-β2 is actually IL-6). IFN-β1 is used as a treatment for multiple sclerosis as it reduces the relapse rate. IFN-β1 is not an appropriate treatment for patients with progressive, non-relapsing forms of multiple sclerosis.

IFN-ε, -κ, -τ, and -ξ appear, at this time, to come in a single isoform in humans, IFNK. Only ruminants encode IFN-τ, a variant of IFN-ω. So far, IFN-ξ is only found in mice, while a structural homolog, IFN-δ is found in a diverse array of non-primate and non-rodent placental mammals. Most but not all placental mammals encode functional IFN-ε and IFN-κ genes.

IFN-ω, although having only one functional form described to date (IFNW1), has several pseudogenes: IFNWP2, IFNWP4, IFNWP5, IFNWP9, IFNWP15, IFNWP18, and IFNWP19 in humans. Many non-primate placental mammals express multiple IFN-ω subtypes

IFN-ν was recently described as a pseudogene in human, but potentially functional in the domestic cat genome. In all other genomes of non-feline placental mammals, IFN-ν is a pseudogene; in some species, the pseudogene is well preserved, while in others, it is badly mutilated or is undetectable. Moreover, in the cat genome, the IFN-ν promoter is deleteriously mutated. It is likely that the IFN-ν gene family was rendered useless prior to mammalian diversification. Its presence on the edge of the Type I IFN locus in mammals may have shielded it from obliteration, allowing its detection.

IFN-α and IFN-β are secreted by many cell types including lymphocytes (NK cells, B-cells and T-cells), macrophages, fibroblasts, endothelial cells, osteoblasts and others. They stimulate both macrophages and NK cells to elicit an anti-viral response, and are also active against tumors. Plasmacytoid dendritic cells have been identified as being the most potent producers of type I IFNs in response to antigen, and have thus been coined natural IFN producing cells. Current study findings suggest that by forcing IFN-α expression in tumor-infiltrating macrophages, it is possible to elicit a more effective dendritic cell activation and immune effector cell cytotoxicity.

IFN-ω is released by leukocytes at the site of viral infection or tumors. IFN-α acts as a pyrogenic factor by altering the activity of thermosensitive neurons in the hypothalamus thus causing fever. It does this by binding to opioid receptors and eliciting the release of prostaglandin-E₂ (PGE₂). A similar mechanism is used by IFN-α to reduce pain; IFN-α interacts with the μ-opioid receptor to act as an analgesic.

In mice, IFN-β inhibits immune cells to produce growth factors, thereby slowing tumor growth, and inhibits other cells from producing vessel producing growth factors, thereby blocking tumor angiogenesis and hindering the tumour from connecting into the blood vessel system.

B. Interferon Receptors

The interferon-α/β receptor (IFNAR) is a receptor which binds type I interferons including interferon-α and -β. It is a heteromeric cell surface receptor composed of one chain with two subunits referred to as IFNAR1 and IFNAR2. Upon binding of type I IFNs, IFNAR activates the JAK-STAT signaling pathway. Interferon stimulation classically results in an anti-viral immune response.

The structure was obtained using NMR. Originally 35 conformers were calculated. This was narrowed to 22 with low energy being the criteria. It was the first helical cytokine receptor's structure to be determined in solution. The molecule has one polymer. The structure reveals the nature of binding. A model of the IFNAR2 reveals a predominantly hydrophobic patch on the receptor that interacts with a matching hydrophobic surface on IFN-alpha. An adjacent motif of charged side chains then guides the proteins into a tight complex. The binding interface may account for crossreactivity and ligand specificity of the receptor. The source for experiments was human but was expressed in Escherichia coli.

IFNAR is clustered into the group beta proteins based on its secondary structural content. IFNAR's folds show a clear evolutionary relationship to the Immunoglobulin beta-sandwich. It is grouped into the superfamily and family Fibronectin type III based on structural and functional similarities that show probable common evolution. The domain, or evolutionary unit observed in nature, is the Interferon-alpha/beta receptor beta chain. Species was human.

### II. INTERFERON PRODRUGS

A. General

Prodrugs are generally defined as molecules that contain the capacity for being therapeutic agents, but in a form requiring some modification in order to actually be therapeutic. Such reagents are particularly useful when delivery of the active drug form has some inherent limitation, such as toxicity or lack of stability. By creating a prodrug form that can both avoid these drawbacks, while permitting effective activation in vivo at the appropriate time and/or site.

The prodrugs of the present disclosure have five distinct components. The first component is the interferon. The second component is a masking domain that blocks the ability of the interferon to exert its normal activities while engaged. The third component is a stabilizing feature, for example, the constant domain on an antibody. These three components are joined by linkers, with the linker disposed between the interferon and the masking domain being subject to selective cleavage, for example, by a protease that is expressed by a cancer cell or in a tumor environment.

As discussed above, the type 1 interferons are IFN-α (alpha), IFN-β (beta), IFN-κ (kappa), IFN-δ (delta), IFN-ε (epsilon), IFN-τ (tau), IFN-ω (omega), and IFN-ξ (zeta). Any of these molecules may be included in the constructs described herein. It is also possible to make heterodimeric constructs with two or even four different type 1 interferons. The interferons used by applicants to generate the data disclosed here were full length proteins but lacking signal peptides.

An important part of the construct is of course the masking domain. The inventors choose to utilize the natural receptors for type 1 interferons rather than select non-natural sequences for this purpose. The advantage of using a masking domain based on the structure of the natural receptors includes both (a) high affinity of the type 1 interferon, and (b) a lower likelihood of immune response against the sequences. The sequences

Mouse IFNAR1-ECD:

| | |
|---|---|
| 1 | mlavvgaaal vlvagapwvl psaaggenlk ppenidvyii |
| | ddnytlkwss hgesmgsvtf |
| 61 | saeyrtkdea kwlkvpecqh ttttkcefsl ldtnvyiktq |
| | frvraeegns tsswnevdpf |
| 121 | ipfytahmsp pevrleaedk ailvhisppg qdgnmwalek |
| | psfsytiriw qksssdkkti |
| 181 | nstyyvekip ellpettycl evkaihpslk khsnystvqc |
| | isttvankmp vpgnlqvdaq |
| 241 | gksyvlkwdy iasadvlfra qwlpgyskss sgsrsdkwkp |
| | iptcanvqtt hcvfsqdtvy |
| 301 | tgtfflhvqa segnhtsfws eekfidsqkh ilppppvitv |
| | tamsdtllvy vncqdstcdg |
| 361 | lnyeiifwen tsntkismek dgpeftlknl qpltvycvqa |
| | rvlfrallnk tsnfseklce |
| 421 | ktrpgsfst |

Mouse IFNAR2-ECD:

| | |
|---|---|
| 1 | mrsrctvsav gllslclvvs asletitpsa fdgypdepct |
| | initirnsrl ilswelenks |
| 61 | gppanytlwy tvmskdenlt kvkncsdttk sscdvtdkwl |
| | egmesyvvai vivhrgdltv |
| 121 | crcsdyivpa napleppefe ivgftdhinv tmefppvtsk |
| | iiqekmkttp fvikeqigds |
| 181 | vrkkhepkvn nvtgnftfvl rdllpktnyc vslyfdddpa |
| | iksplkcivl qpgqesglse |
| 241 | sa |

Human IFNAR1-ECD:

| | |
|---|---|
| 1 | mmvvllgatt lvlvavapwv lsaaaggknl kspqkvevdi |
| | iddnfilrwn rsdesvgnvt |
| 61 | fsfdyqktgm dnwiklsgcq nitstkcnfs slklnvyeei |
| | klriraeken tsswyevdsf |
| 121 | tpfrkaqigp pevhleaedk aivihispgt kdsvmwaldg |
| | lsftyslviw knssgveeri |
| 181 | eniysrhkiy klspettycl kvkaalltsw kigvyspvhc |
| | ikttvenelp ppenievsvq |
| 241 | nqnyvlkwdy tyanmtfqvq wlhaflkrnp gnhlykwkqi |
| | pdcenvkttq cvfpqnvfqk |
| 301 | giyllrvqas dgnntsfwse eikfdteiqa fllppvfnir |
| | slsdsfhiyi gapkqsgntp |
| 361 | viqdypliye iifwentsna erkiiekktd vtvpnlkplt |
| | vycvkaraht mdeklnkssv |
| 421 | fsdavcektk pgntsk |

Human IFNAR2-ECD:

| | |
|---|---|
| 1 | mllsqnafif rslnlvlmvy islvfgisyd spdytdesct |
| | fkislrnfrs ilswelknhs |
| 61 | ivpthytlly timskpedlk vvkncanttr sfcdltdewr |
| | stheayvtvl egfsgnttlf |
| 121 | scshnfwlai dmsfeppefe ivgftnhinv mvkfpsivee |
| | elqfdlslvi eeqsegivkk |
| 181 | hkpeikgnms gnftyiidkl ipntnycvsv ylehsdeqav |
| | iksplkctll ppgqesesae |
| 241 | sak |

Next, the constructs include stabilization domains that, for example, will increase half-life in vivo. The inventors chose to employ an Ig constant domain. While the IgG1 Fc domain was employed, other Fc domains may also be used. A particular advantage to the use of IgA Fc domains is their ability to dimerize, meaning that up to four different interferons could be included in such a construct. For creating multiple interferon constructs, one example would be to use a FcA-FcB heterodimer of IgG1, with one blocking receptor and one interferon fused to FcA or FcB, while using distinct cleavage substrates specific for different enzymes inserted in the linkers of FcA and FcB, respectively. Other stabilizing proteins could also be used, including human serum albumin and transferrin.

Finally, the domains above are linked together by short peptide stretches, or “linkers.” One of these linkers, the one disposed between the interferon molecule and the masking domain, is subject to cleavage when the prodrug arrives at or near its target—a tumor or cancer cell. In a particular, embodiment, the linker contains a protease target site such that when the prodrug is exposed to an environment containing the protease, the linker is cleaved and the masking agent is released from the prodrug, thereby activating the interferon molecule. Ideally, the linker can be selected for proteases overexpressed in cancer cells or at a tumor site, and may be even be tailored to an particular individual's cancer/tumor protease profile by prior testing. Below are a few examples of enzymes selectively expressed or overexpressed in cancers or tumor environments.

TABLE 2
PROTEASE SUBSTRATES

| Enzyme | Substrate amino acid sequence |
|---|---|
| MMP2, MMP9 | PVGLIG |
| MMP11 | GGYAELRMGG |
| MMP13 | GPRPFNYL |
| MMP13 | GGALGLSL |
| MMP13 | GPMSYNAL |
| MMP14 | SGRSENIRTA |
| FAPa | TSGPNQEQK |
| Cathepsin B | GFLG |

An exemplary form of the linker is GGGGS-substrate-GGGGS. Other two examples are (GGGGS)ₙ-substrate-(GGGGS)ₙ, and Gₙ-substrate-Gₙ (n could be any number).

B. Engineering of and Expression of Nucleic Acid Constructs

Various genetic constructs are available that contain the interferon prodrug components described above, and these may be introduced into vectors for expression. Nucleic acids according to the present disclosure that encode prodrug molecules may be optionally linked to other protein sequences. Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al. (1989) and Ausubel et al. (1994), both incorporated herein by reference.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

1. Regulatory Elements

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally-associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment.

A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally-occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202, 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. (1989), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

The identity of tissue-specific promoters or elements, as well as assays to characterize their activity, is well known to those of skill in the art. Examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), DIA dopamine receptor gene (Lee, et al., 1997), insulin-like growth factor II (Wu et al., 1997), human platelet endothelial cell adhesion molecule-1 (Almendro et al., 1996). Tumor specific promoters also will find use in the present disclosure.

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

2. IRES

In certain embodiments of the disclosure, the use of internal ribosome entry sites (IRES) elements are used to create multigene, or polycistronic, messages. IRES elements are able to bypass the ribosome scanning model of 5′-methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

3. Multi-Purpose Cloning Sites

Vectors can include a multiple cloning site (MCS), which is a nucleic acid region that contains multiple restriction enzyme sites, any of which can be used in conjunction with standard recombinant technology to digest the vector. See Carbonelli et al., 1999, Levenson et al., 1998, and Cocea, 1997, incorporated herein by reference. “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

4. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see Chandler et al., 1997, herein incorporated by reference).

5. Termination Signals

The vectors or constructs of the present disclosure will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and/or to minimize read through from the cassette into other sequences.

Terminators contemplated for use in the disclosure include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

6. Polyadenylation Signals

In expression, particularly eukaryotic expression, one will typically include a polyadenylation signal to effect proper polyadenylation of the transcript. The nature of the polyadenylation signal is not believed to be crucial to the successful practice of the disclosure, and/or any such sequence may be employed. Preferred embodiments include the SV40 polyadenylation signal and/or the bovine growth hormone polyadenylation signal, convenient and/or known to function well in various target cells. Polyadenylation may increase the stability of the transcript or may facilitate cytoplasmic transport.

7. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

8. Selectable and Screenable Markers

In certain embodiments of the disclosure, cells containing a nucleic acid construct of the present disclosure may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

9. Viral Vectors

The capacity of certain viral vectors to efficiently infect or enter cells, to integrate into a host cell genome and stably express viral genes, have led to the development and application of a number of different viral vector systems (Robbins et al., 1998). Viral systems are currently being developed for use as vectors for ex vivo and in vivo gene transfer. For example, adenovirus, herpes-simplex virus, retrovirus and adeno-associated virus vectors are being evaluated currently for treatment of diseases such as cancer, cystic fibrosis, Gaucher's disease, renal disease and arthritis (Robbins and Ghivizzani, 1998; Imai et al., 1998; U.S. Pat. No. 5,670,488). The various viral vectors described below, present specific advantages and disadvantages, depending on the particular gene-therapeutic application.

10. Non-Viral Transformation

Suitable methods for nucleic acid delivery for transformation of an organelle, a cell, a tissue or an organism for use with the current disclosure are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985). Through the application of techniques such as these, organelle(s), cell(s), tissue(s) or organism(s) may be stably or transiently transformed.

Injection.

In certain embodiments, a nucleic acid may be delivered to an organelle, a cell, a tissue or an organism via one or more injections (i.e., a needle injection), such as, for example, either subcutaneously, intradermally, intramuscularly, intervenously or intraperitoneally. Methods of injection of vaccines are well known to those of ordinary skill in the art (e.g., injection of a composition comprising a saline solution). Further embodiments of the present disclosure include the introduction of a nucleic acid by direct microinjection. Direct microinjection has been used to introduce nucleic acid constructs into Xenopus oocytes (Harland and Weintraub, 1985).

Electroporation.

In certain embodiments of the present disclosure, a nucleic acid is introduced into an organelle, a cell, a tissue or an organism via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding.

Transfection of eukaryotic cells using electroporation has been quite successful. Mouse pre-B lymphocytes have been transfected with human κ-immunoglobulin genes (Potter et al., 1984), and rat hepatocytes have been transfected with the chloramphenicol acetyltransferase gene (Tur-Kaspa et al., 1986) in this manner.

To effect transformation by electroporation in cells such as, for example, plant cells, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wounding in a controlled manner. Examples of some species which have been transformed by electroporation of intact cells include maize (U.S. Pat. No. 5,384,253; Rhodes et al., 1995; D'Halluin et al., 1992), wheat (Zhou et al., 1993), tomato (Hou and Lin, 1996), soybean (Christou et al., 1987) and tobacco (Lee et al., 1989).

One also may employ protoplasts for electroporation transformation of plant cells (Bates, 1994; Lazzeri, 1995). For example, the generation of transgenic soybean plants by electroporation of cotyledon-derived protoplasts is described by Dhir and Widholm in International Patent Application No. WO 92/17598, incorporated herein by reference. Other examples of species for which protoplast transformation has been described include barley (Lazerri, 1995), sorghum (Battraw et al., 1991), maize (Bhattacharjee et al., 1997), wheat (He et al., 1994) and tomato (Tsukada, 1989).

Calcium Phosphate.

In other embodiments of the present disclosure, a nucleic acid is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

DEAE-Dextran.

In another embodiment, a nucleic acid is delivered into a cell using DEAE-dextran followed by polyethylene glycol. In this manner, reporter plasmids were introduced into mouse myeloma and erythroleukemia cells (Gopal, 1985).

Sonication Loading.

Additional embodiments of the present disclosure include the introduction of a nucleic acid by direct sonic loading. LTK⁻ fibroblasts have been transfected with the thymidine kinase gene by sonication loading (Fechheimer et al., 1987).

Liposome-Mediated Transfection.

In a further embodiment of the disclosure, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an nucleic acid complexed with Lipofectamine (Gibco BRL) or Superfect (Qiagen).

Liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980).

In certain embodiments of the disclosure, a liposome may be complexed with a hemagglutinating virus (HVJ). This has been shown to facilitate fusion with the cell membrane and promote cell entry of liposome-encapsulated DNA (Kaneda et al., 1989). In other embodiments, a liposome may be complexed or employed in conjunction with nuclear non-histone chromosomal proteins (HMG-1) (Kato et al., 1991). In yet further embodiments, a liposome may be complexed or employed in conjunction with both HVJ and HMG-1. In other embodiments, a delivery vehicle may comprise a ligand and a liposome.

Receptor-Mediated Transfection.

Still further, a nucleic acid may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity to the present disclosure.

Certain receptor-mediated gene targeting vehicles comprise a cell receptor-specific ligand and a nucleic acid-binding agent. Others comprise a cell receptor-specific ligand to which the nucleic acid to be delivered has been operatively attached. Several ligands have been used for receptor-mediated gene transfer (Wu and Wu, 1987; Wagner et al., 1990; Perales et al., 1994; Myers, EPO 0273085), which establishes the operability of the technique. Specific delivery in the context of another mammalian cell type has been described (Wu and Wu, 1993; incorporated herein by reference). In certain aspects of the present disclosure, a ligand will be chosen to correspond to a receptor specifically expressed on the target cell population.

In other embodiments, a nucleic acid delivery vehicle component of a cell-specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the nucleic acid delivery vehicle component of a targeted delivery vehicle may be a liposome itself, which will preferably comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al., 1987). It is contemplated that the tissue-specific transforming constructs of the present disclosure can be specifically delivered into a target cell in a similar manner.

11. Expression Systems

Numerous expression systems exist that comprise at least a part or all of the compositions discussed above. Prokaryote- and/or eukaryote-based systems can be employed for use with the present disclosure to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986 and 4,879,236, both herein incorporated by reference, and which can be bought, for example, under the name MaxBac® 2.0 from Invitrogen® and BacPack™ Baculovirus Expression System From Clontech®.

Other examples of expression systems include Stratagene®'s Complete Control™ Inducible Mammalian Expression System, which involves a synthetic ecdysone-inducible receptor, or its pET Expression System, an E. coli expression system. Another example of an inducible expression system is available from Invitrogen®, which carries the T-Rex™ (tetracycline-regulated expression) System, an inducible mammalian expression system that uses the full-length CMV promoter. Invitrogen® also provides a yeast expression system called the Pichia methanolica Expression System, which is designed for high-level production of recombinant proteins in the methylotrophic yeast Pichia methanolica. pGEM-T Easy vectors, pCon Vectors™, Lonza pConIgG1 or pConK2 plasmid vectors, and 293 Freestyle cells or Lonza CHO cells are also useful for expression of the disclosed prodrug constructs.

Primary mammalian cell cultures may be prepared in various ways. In order for the cells to be kept viable while in vitro and in contact with the expression construct, it is necessary to ensure that the cells maintain contact with the correct ratio of oxygen and carbon dioxide and nutrients but are protected from microbial contamination. Cell culture techniques are well documented.

One embodiment of the foregoing involves the use of gene transfer to immortalize cells for the production of proteins. The gene for the protein of interest may be transferred as described above into appropriate host cells followed by culture of cells under the appropriate conditions. The gene for virtually any polypeptide may be employed in this manner. The generation of recombinant expression vectors, and the elements included therein, are discussed above. Alternatively, the protein to be produced may be an endogenous protein normally synthesized by the cell in question.

Examples of useful mammalian host cell lines are Vero and HeLa cells and cell lines of Chinese hamster ovary, W138, BHK, COS-7, 293, HepG2, NIH3T3, RIN and MDCK cells. In addition, a host cell strain may be chosen that modulates the expression of the inserted sequences, or modifies and process the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to insure the correct modification and processing of the foreign protein expressed.

A number of selection systems may be used including, but not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection for dhfr that confers resistance to; gpt, which confers resistance to mycophenolic acid; neo, that confers resistance to the aminoglycoside G418; and hygro, which confers resistance to hygromycin.

E. Purification

In certain embodiments, the interferon prodrugs of the present disclosure may be purified. The term “purified,” as used herein, is intended to refer to a composition, isolatable from other components, wherein the protein is purified to any degree relative to its naturally-obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxylapatite and affinity chromatography; and combinations of such and other techniques.

In purifying an interferon prodrug of the present disclosure, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide. Commonly, antibodies are fractionated utilizing agents (i.e., protein A) that bind the Fc portion of the antibody. Where the interferon prodrug contains such a domain, this approach could be used.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

### III. PHARMACEUTICAL FORMULATIONS AND TREATMENT OF CANCER

A. Cancers

Cancer results from the outgrowth of a clonal population of cells from tissue. The development of cancer, referred to as carcinogenesis, can be modeled and characterized in a number of ways. An association between the development of cancer and inflammation has long-been appreciated. The inflammatory response is involved in the host defense against microbial infection, and also drives tissue repair and regeneration. Considerable evidence points to a connection between inflammation and a risk of developing cancer, i.e., chronic inflammation can lead to dysplasia.

Cancer cells to which the methods of the present disclosure can be applied include generally any cell that selectively express proteases, and more particularly, that overexpresses such proteases compared to normal cells. An appropriate cancer cell can be a breast cancer, lung cancer, colon cancer, pancreatic cancer, renal cancer, stomach cancer, liver cancer, bone cancer, hematological cancer (e.g., leukemia or lymphoma), neural tissue cancer, melanoma, ovarian cancer, testicular cancer, prostate cancer, cervical cancer, vaginal cancer, or bladder cancer cell. In addition, the methods of the disclosure can be applied to a wide range of species, e.g., humans, non-human primates (e.g., monkeys, baboons, or chimpanzees), horses, cattle, pigs, sheep, goats, dogs, cats, rabbits, guinea pigs, gerbils, hamsters, rats, and mice. Cancers may also be recurrent, metastatic and/or multi-drug resistant, and the methods of the present disclosure may be particularly applied to such cancers so as to render them resectable, to prolong or re-induce remission, to inhibit angiogenesis, to prevent or limit metastasis, and/or to treat multi-drug resistant cancers. At a cellular level, this may translate into killing cancer cells, inhibiting cancer cell growth, or otherwise reversing or reducing the malignant phenotype of tumor cells.

B. Formulation and Administration

The present disclosure provides pharmaceutical compositions comprising interferon prodrugs. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Other suitable pharmaceutical excipients include starch, glucose, lactose, sucrose, saline, dextrose, gelatin, malt, rice, flour, chalk, silica gel, sodium stearate, glycerol monostearate, talc, sodium chloride, dried skim milk, glycerol, propylene glycol, water, ethanol and the like.

The compositions can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The antibodies of the present disclosure may include classic pharmaceutical preparations. Administration of these compositions according to the present disclosure will be via any common route so long as the target tissue is available via that route. This includes oral, nasal, buccal, rectal, vaginal or topical. Alternatively, administration may be by intradermal, subcutaneous, intramuscular, intraperitoneal or intravenous injection. Such compositions would normally be administered as pharmaceutically acceptable compositions, described supra. Of particular interest is direct intratumoral administration, perfusion of a tumor, or administration local or regional to a tumor, for example, in the local or regional vasculature or lymphatic system, or in a resected tumor bed.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

C. Combination Therapies

In the context of the present disclosure, it also is contemplated that interferon prodrugs described herein could be used similarly in conjunction with immuno-, chemo- or radiotherapeutic intervention, or other treatments. It also may prove effective, in particular, to combine interferon prodrugs with other therapies that target different aspects of cancer cell function.

To kill cells, inhibit cell growth, inhibit metastasis, inhibit angiogenesis or otherwise reverse or reduce the malignant phenotype of tumor cells, using the methods and compositions of the present disclosure, one would generally contact a “target” cell with an interferon prodrugs according to the present disclosure and at least one other agent. These compositions would be provided in a combined amount effective to kill or inhibit proliferation of the cell. This process may involve contacting the cells with the interferon prodrugs according to the present disclosure and the other agent(s) or factor(s) at the same time. This may be achieved by contacting the cell with a single composition or pharmacological formulation that includes both agents, or by contacting the cell with two distinct compositions or formulations, at the same time, wherein one composition includes the interferon prodrugs according to the present disclosure and the other includes the other agent.

Alternatively, the interferon prodrugs therapy may precede or follow the other agent treatment by intervals ranging from minutes to weeks. In embodiments where the other agent and the interferon prodrugs are applied separately to the cell, one would generally ensure that a significant period of time did not expire between each delivery, such that both agents would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either interferon prodrugs or the other agent will be desired. Various combinations may be employed, where an interferon prodrug according to the present disclosure is “A” and the other therapy is “B”, as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated. Again, to achieve cell killing, both agents are delivered to a cell in a combined amount effective to kill the cell.

Agents or factors suitable for cancer therapy include any chemical compound or treatment method that induces DNA damage when applied to a cell. Such agents and factors include radiation and waves that induce DNA damage such as, irradiation, microwaves, electronic emissions, and the like. A variety of chemical compounds, also described as “chemotherapeutic” or “genotoxic agents,” may be used. This may be achieved by irradiating the localized tumor site; alternatively, the tumor cells may be contacted with the agent by administering to the subject a therapeutically effective amount of a pharmaceutical composition.

Various classes of chemotherapeutic agents are comtemplated for use with the present disclosure. For example, selective estrogen receptor antagonists (“SERMs”), such as Tamoxifen, 4-hydroxy Tamoxifen (Afimoxfene), Falsodex, Raloxifene, Bazedoxifene, Clomifene, Femarelle, Lasofoxifene, Ormeloxifene, and Toremifene.

Chemotherapeutic agents contemplated to be of use, include, e.g., camptothecin, actinomycin-D, mitomycin C. The disclosure also encompasses the use of a combination of one or more DNA damaging agents, whether radiation-based or actual compounds, such as the use of X-rays with cisplatin or the use of cisplatin with etoposide. The agent may be prepared and used as a combined therapeutic composition.

Heat shock protein 90 is a regulatory protein found in many eukaryotic cells. HSP90 inhibitors have been shown to be useful in the treatment of cancer. Such inhibitors include Geldanamycin, 17-(Allylamino)-17-demethoxygeldanamycin, PU-H71 and Rifabutin.

Agents that directly cross-link DNA or form adducts are also envisaged. Agents such as cisplatin, and other DNA alkylating agents may be used. Cisplatin has been widely used to treat cancer, with efficacious doses used in clinical applications of 20 mg/m² for 5 days every three weeks for a total of three courses. Cisplatin is not absorbed orally and must therefore be delivered via injection intravenously, subcutaneously, intratumorally or intraperitoneally.

Agents that damage DNA also include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 mg/m² at 21 day intervals for doxorubicin, to 35-50 mg/m² for etoposide intravenously or double the intravenous dose orally. Microtubule inhibitors, such as taxanes, also are contemplated. These molecules are diterpenes produced by the plants of the genus Taxus, and include paclitaxel and docetaxel.

Epidermal growth factor receptor inhibitors, such as Iressa, mTOR, the mammalian target of rapamycin, also known as FK506-binding protein 12-rapamycin associated protein 1 (FRAP1) is a serine/threonine protein kinase that regulates cell growth, cell proliferation, cell motility, cell survival, protein synthesis, and transcription. Rapamycin and analogs thereof (“rapalogs”) are therefore contemplated for use in cancer therapy in accordance with the present disclosure.

Another possible therapy is TNF-α (tumor necrosis factor-alpha), a cytokine involved in systemic inflammation and a member of a group of cytokines that stimulate the acute phase reaction. The primary role of TNF is in the regulation of immune cells. TNF is also able to induce apoptotic cell death, to induce inflammation, and to inhibit tumorigenesis and viral replication.

Agents that disrupt the synthesis and fidelity of nucleic acid precursors and subunits also lead to DNA damage. As such a number of nucleic acid precursors have been developed. Particularly useful are agents that have undergone extensive testing and are readily available. As such, agents such as 5-fluorouracil (5-FU), are preferentially used by neoplastic tissue, making this agent particularly useful for targeting to neoplastic cells. Although quite toxic, 5-FU, is applicable in a wide range of carriers, including topical, however intravenous administration with doses ranging from 3 to 15 mg/kg/day being commonly used.

Other factors that cause DNA damage and have been used extensively include what are commonly known as y-rays, x-rays, and/or the directed delivery of radioisotopes to tumor cells. Other forms of DNA damaging factors are also contemplated such as microwaves and UV-irradiation. It is most likely that all of these factors effect a broad range of damage DNA, on the precursors of DNA, the replication and repair of DNA, and the assembly and maintenance of chromosomes. Dosage ranges for x-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

In addition, it also is contemplated that immunotherapy, hormone therapy, toxin therapy and surgery can be used. In particular, one may employ targeted therapies such as Avastin, Erbitux, Gleevec, Herceptin and Rituxan.

The skilled artisan is directed to “Remington's Pharmaceutical Sciences” 15th Edition, Chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### IV. PROTEASE DETECTION METHODS

In still further embodiments, it will be desireable to identify the nature and quantity of protease expression in a target cancer cell or tumor, thereby permitting one to tailor a specific interferon prodrug that will have a high likelihood of activation by the patient's tumor. In general, there are three approaches to such detection—immunologic detection, such as with protease-specific antibodies, mRNA detection, and protease activity assays.

A. Immunodetection

Immunodetection methods for identifying and quantifying proteases may include enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle and Ben-Zeev (1999), Gulbis and Galand (1993), De Jager et al. (1993), and Nakamura et al. (1987). In general, the immunobinding methods include obtaining a sample and contacting the sample with a first antibody in accordance with embodiments discussed herein, as the case may be, under conditions effective to allow the formation of immunocomplexes.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to protease present. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. Patents concerning the use of such labels include U.S. Pat. Nos. 3,817,837, 3,850,752, 3,939,350, 3,996,345, 4,277,437, 4,275,149 and 4,366,241. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody that has binding affinity for the antibody, is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first biotinylated antibody is used to detect the target antigen, and a second antibody is then used to detect the biotin attached to the complexed biotin. In that method, the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

In one exemplary ELISA, the antibodies of the disclosure are immobilized onto a selected surface exhibiting protein affinity, such as a well in a polystyrene microtiter plate. Then, a test composition suspected of containing the protease is added to the wells. After binding and washing to remove non-specifically bound immune complexes, the bound antigen may be detected. Detection may be achieved by the addition of another anti-protease antibody that is linked to a detectable label. This type of ELISA is a simple “sandwich ELISA.” Detection may also be achieved by the addition of a second anti-protease antibody, followed by the addition of a third antibody that has binding affinity for the second antibody, with the third antibody being linked to a detectable label.

In another exemplary ELISA, the samples suspected of containing the protease are immobilized onto the well surface and then contacted with anti-protease antibody. After binding and washing to remove non-specifically bound immune complexes, the bound anti-protease antibodies are detected. Where the initial protease antibodies are linked to a detectable label, the immune complexes may be detected directly. Again, the immune complexes may be detected using a second antibody that has binding affinity for the first anti-protease antibody, with the second antibody being linked to a detectable label.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

B. mRNA Detection

mRNA detecting can be used to assess protease activity in a cancer cell or tumor. In general, mRNA detection relies on hybridization of one nucleic acid—a probe or primer—to another nucleic acid (the target).

The use of a probe or primer of between 13 and 100 nucleotides, preferably between 17 and 100 nucleotides in length, or in some aspects up to 1-2 kilobases or more in length, allowing the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, nucleotide sequences may be used for their ability to selectively form duplex molecules with complementary stretches mRNAs or to provide primers for amplification of mRNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications it is appreciated that lower stringency conditions are preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR™, for detection of expression of corresponding genes, as well as in embodiments employing a solid phase. In embodiments involving a solid phase, the test mRNA is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772 and U.S. Patent Publication 2008/0009439. The relevant portions of these and other references identified in this section of the Specification are incorporated herein by reference.

Target nucleic acids may be isolated from cells, tissues or other samples according to standard methodologies (Sambrook et al., 2001). In certain embodiments, analysis is performed on whole cell or tissue homogenates or biological fluid samples without substantial purification of the template nucleic acid. In other cases, purification and/or amplification may be required.

Amplification typically involves pairs of primers designed to selectively hybridize to nucleic acids corresponding to a target under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Bellus, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety.

A reverse transcriptase PCR™ amplification procedure may be performed to quantify the amount of mRNA amplified. Methods of reverse transcribing RNA into cDNA are well known (see Sambrook et al., 2001). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Reverse transcription (RT) of RNA to cDNA followed by quantitative PCR (RT-PCR) can be used to determine the relative concentrations of specific mRNA species isolated from a cell. By determining that the concentration of a specific mRNA species varies, it is shown that the gene encoding the specific mRNA species is differentially expressed. If a graph is plotted in which the cycle number is on the X axis and the log of the concentration of the amplified target DNA is on the Y axis, a curved line of characteristic shape is formed by connecting the plotted points. Beginning with the first cycle, the slope of the line is positive and constant. This is said to be the linear portion of the curve. After a reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR amplification is directly proportional to the starting concentration of the target before the reaction began. By determining the concentration of the amplified products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived can be determined for the respective tissues or cells. This direct proportionality between the concentration of the PCR products and the relative mRNA abundances is only true in the linear range of the PCR reaction.

The final concentration of the target DNA in the plateau portion of the curve is determined by the availability of reagents in the reaction mix and is independent of the original concentration of target DNA. Therefore, the first condition that must be met before the relative abundances of a mRNA species can be determined by RT-PCR for a collection of RNA populations is that the concentrations of the amplified PCR products must be sampled when the PCR reactions are in the linear portion of their curves.

A second condition for an RT-PCR experiment is to determine the relative abundances of a particular mRNA species. Typically, relative concentrations of the amplifiable cDNAs are normalized to some independent standard. The goal of an RT-PCR experiment is to determine the abundance of a particular mRNA species relative to the average abundance of all mRNA species in the sample.

Most protocols for competitive PCR utilize internal PCR standards that are approximately as abundant as the target. These strategies are effective if the products of the PCR amplifications are sampled during their linear phases. If the products are sampled when the reactions are approaching the plateau phase, then the less abundant product becomes relatively over represented. Comparisons of relative abundances made for many different RNA samples, such as is the case when examining RNA samples for differential expression, become distorted in such a way as to make differences in relative abundances of RNAs appear less than they actually are. This is not a significant problem if the internal standard is much more abundant than the target. If the internal standard is more abundant than the target, then direct linear comparisons can be made between RNA samples.

RT-PCR can be performed as a relative quantitative RT-PCR with an internal standard in which the internal standard is an amplifiable cDNA fragment that is larger than the target cDNA fragment and in which the abundance of the mRNA encoding the internal standard is roughly 5-100 fold higher than the mRNA encoding the target. This assay measures relative abundance, not absolute abundance of the respective mRNA species.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assy (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1990; Ohara et al., 1989).

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 2001). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 2001). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Various nucleic acid detection methods known in the art are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

Protease mRNA detection may involve the use of arrays or data generated from an array. An array generally refers to ordered macroarrays or microarrays of nucleic acid molecules (probes) that are fully or nearly complementary or identical to a plurality of mRNA molecules or cDNA molecules and that are positioned on a support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g., up to about 100 or even 1000 per square centimeter. Microarrays typically use coated glass as the solid support, in contrast to the nitrocellulose-based material of filter arrays. By having an ordered array of complementing nucleic acid samples, the position of each sample can be tracked and linked to the original sample. A variety of different array devices in which a plurality of distinct nucleic acid probes are stably associated with the surface of a solid support are known to those of skill in the art. Useful substrates for arrays include nylon, glass and silicon Such arrays may vary in a number of different ways, including average probe length, sequence or types of probes, nature of bond between the probe and the array surface, e.g., covalent or non-covalent, and the like. The labeling and screening methods of the present invention and the arrays are not limited in its utility with respect to any parameter except that the probes detect expression levels; consequently, methods and compositions may be used with a variety of different types of genes.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610,287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; WO0138580; WO 0168255; WO 03020898; WO 03040410; WO 03053586; WO 03087297; WO 03091426; WO03100012; WO 04020085; WO 04027093; EP 373 203; EP 785 280; EP 799 897 and UK 8 803 000; the disclosures of which are all herein incorporated by reference.

It is contemplated that the arrays can be high density arrays, such that they contain 100 or more different probes. It is contemplated that they may contain 1000, 16,000, 65,000, 250,000 or 1,000,000 or more different probes. The probes can be directed to targets in one or more different organisms. The oligonucleotide probes range from 5 to 50, 5 to 45, 10 to 40, or 15 to 40 nucleotides in length in some embodiments. In certain embodiments, the oligonucleotide probes are 20 to 25 nucleotides in length.

The location and sequence of each different probe sequence in the array are generally known. Moreover, the large number of different probes can occupy a relatively small area providing a high density array having a probe density of generally greater than about 60, 100, 600, 1000, 5,000, 10,000, 40,000, 100,000, or 400,000 different oligonucleotide probes per cm². The surface area of the array can be about or less than about 1, 1.6, 2, 3, 4, 5, 6, 7, 8, 9, or 10 cm².

Moreover, a person of ordinary skill in the art could readily analyze data generated using an array. Such protocols are disclosed above, and include information found in WO 9743450; WO 03023058; WO 03022421; WO 03029485; WO 03067217; WO 03066906; WO 03076928; WO 03093810; WO 03100448A1, all of which are specifically incorporated by reference.

### V. EXAMPLES

The following examples are included to demonstrate preferred embodiments. It should be appreciated by those of skill in the art that the techniques disclosed in the examples which follow represent techniques discovered by the inventors to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

IFN-Prodrug Based on Interferon Alpha and Beta Receptor (IFNAR).

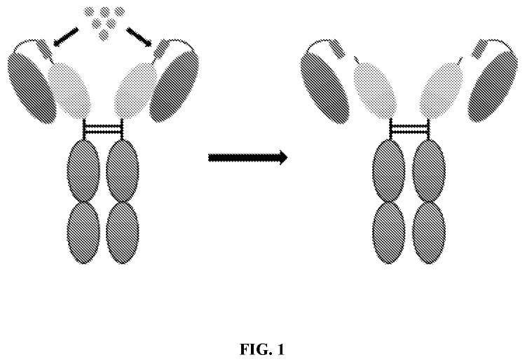

The inventors engineered the IFN-Prodrug based on the structure of human immunoglobulin G1 (IgG1) (FIG. 1). The blocking reagent is the extracellular domain (ECD) of either natural IFNAR1 or IFNAR2, and is linked to the N-terminus of interferon alpha by a cleavable substrate linker. In this form, IFNAR blocks binding to cell surface IFNAR1/IFNAR2 heterodimer. To increase the stability and half-life, the inventors fused the IgG1 Fc domain to the C-terminus of interferon alpha (FIG. 1).

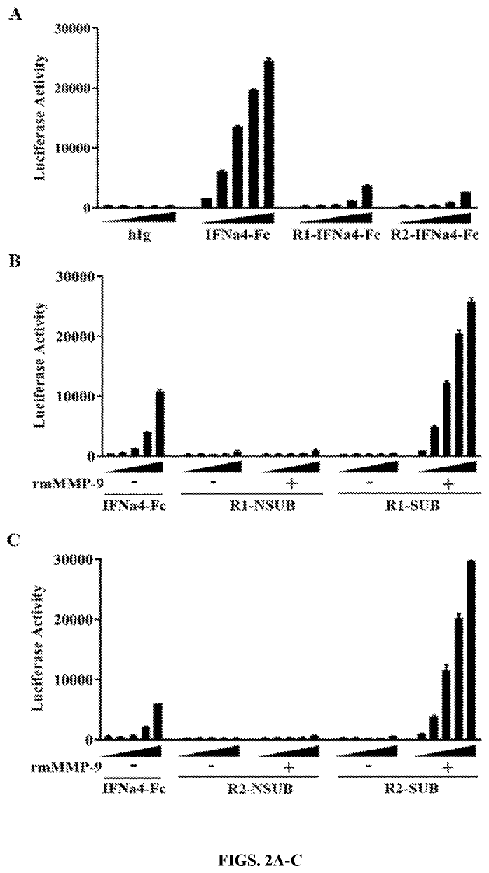

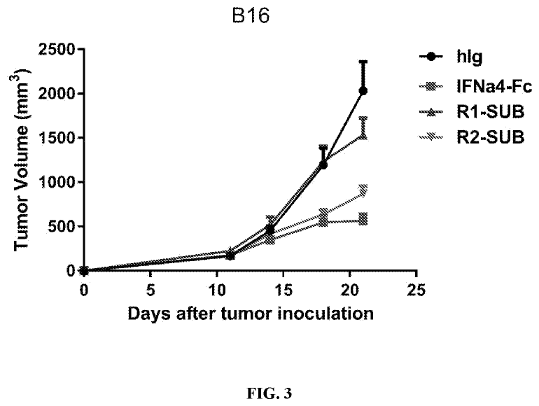

To determine whether the ECD of either IFNAR1 or IFNAR2 is capable of blocking interferon alpha activity, the inventors purified IFNa4-Fc, R1-IFNa4-Fc, and R2-IFNa4-Fc. The linker of R1-IFNa4-Fc or R2-IFNa4-Fc is 15-amino acid of triple Gly-Gly-Gly-Gly-Ser peptide, which is a flexible liker allowing for interaction between domains²³. They used RAW-Lucia ISG cells which express the Lucia luciferase gene under the control of an ISG54 minimal promoter in conjunction with five IFN-stimulated response elements. RAW-Lucia ISG cells are responsive to murine IFN-α and IFN-β. hIg, IFNa4-Fc, R1-IFNa4-Fc, and R2-IFNa4-Fc were serial-diluted in 5 fold from 20 nM for luciferase reporter assay. Both R1-IFNa4-Fc and R2-IFNa4-Fc show significant decrease of IFN activity by at least 125-fold relative to that of IFNa4-Fc (FIG. 2A).

To determine whether the activity of the activated IFN-Prodrug was comparable to that of IFNa4-Fc, the inventors purified R1-NSUB, R1-SUB, R2-NSUB and R2-SUB. The linker of R1-SUB or R2-SUB is a 16-amino acid peptide carrying the 6-residue protease-cleavable substrate (PVGLIG)²⁴, which can be cut by MMP-2 or MMP-9, flanked by flexible Gly-Gly-Gly-Gly-Ser peptides on both sides. The R1-NSUB and R2-NSUB without protease-cleavable substrate were used as control constructs. R1-NSUB, R1-SUB, R2-NSUB or R2-SUB were incubated with rmMMP-9 at 37° C. for 6 hours for activation. Activated R1-SUB showed increased IFN activity by 25-fold relative to that of IFNa4-Fc, while the activity of R1-NSUB did not change after enzyme inoculation (FIG. 2B). A similar result was observed with R2-NSUB and R2-SUB, while activated R2-SUB showed even greater increased IFN activity, more than 25-fold relative to that of IFNa4-Fc (FIG. 2C).

Thus, the biological activity of protease-activated IFN-Prodrug was increased in both IFNA1-based or IFNAR2-based forms, rather than the expected restoration to equivalence to that the parental IFNa4-Fc. Moreover, the IFN-Prodrug appeared remarkably reduced in its biological activity prior to protease cleavage, suggesting that the IFN-Prodrug would remain safe in vivo until cleaved, and thereafter increasing in therapeutic activity at the local tumor microenvironment.

IFNAR1-Based and IFNAR2-Based IFN-Prodrug Show Varied Anti-Tumor Effects In Vivo.

The inventors next investigated whether local activation of IFN-Prodrug would convert into anti-tumor efficacy in vivo using the B16 melanoma model in mice. Without screening enzyme expression in mouse tumor models, they initially started with and IFN-Prodrug carrying the cleavable substrate of MMP-2 and MMP-9. Mice with established B16 melanoma tumors were treated with R1-SUB, R2-SUB, IFNa4-Fc, or hIg control. At a dosing of 1 nmol (three injections for every three days), R1-SUB, R2-SUB, and IFNa4-Fc demonstrated varied efficacy at suppressing tumor growth in this model. At a time point 10 days after treatment, R1-SUB had inhibited tumor growth by 24%, R2-SUB by 57%, and IFNa4-Fc by 72% relative to the hIg-treated control group (FIG. 3).

Therefore, IFNAR2-based IFN-Prodrug showed better anti-tumor effects than IFNAR1-based IFN-Prodrug in vivo, leading the inventors to focus on R2-SUB in the following study. However, R2-SUB carrying cleavable substrate of MMP-2 and MMP-9 had compromised efficacy in suppressing tumor growth relative to that of IFNa4-Fc. The inventors hypothesized that improved efficacy of the IFN-Prodrug will be achieved by selecting an appropriate cleavable substrate linker in mouse tumor models.

IFNAR2-Based IFN-Prodrug has Improved Efficacy and Reduced Side Effects.

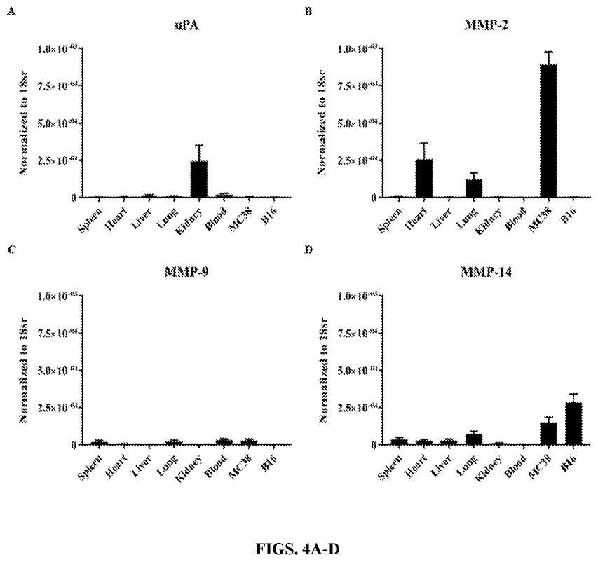

To choose a more advantageous cleavable substrate linker, the inventors screened the most studied proteases known to be up-regulated in a variety of human cancers in mouse tumor models. This substrate selection process included counter-selection against proteases expressed in normal healthy tissues to reduce the potential for systemic (non-specific) activation of the IFN-Prodrug. Mice with established B16 melanoma tumors or MC38 colon tumors were sacrificed and tumor tissues and normal tissues including spleen, heart, liver, lung, and kidney were used to determine the mRNA expression level of following proteases genes: uPA, MMP-2, MMP-9, and MMP-14. UPA was only highly expressed in kidney, but not in two tumor tissues (FIG. 4A). MMP-2 was highly expressed in MC38 tumors, but not B16 tumors; however, it had high background in heart and lung (FIG. 4B). The expression level of MMP-9 was very low in all the normal and tumor tissues (FIG. 4C). Finally, both MC38 and B16 tumors had high MMP-14 expression, while in contrast, MMP-14 expression in normal tissues in minimal (FIG. 4D). Screening data in mouse tumor models suggested that a cleavable substrate linker which is sensitive to MMP-14 would make for a potent IFN-Prodrug in vivo.

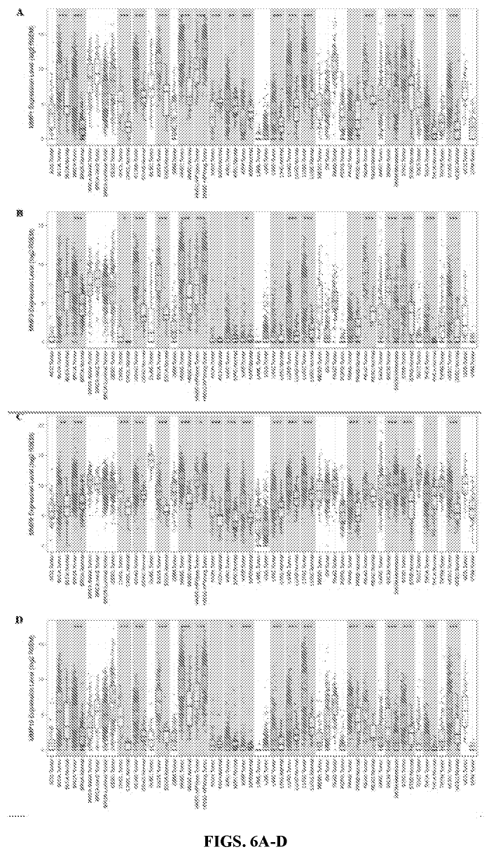

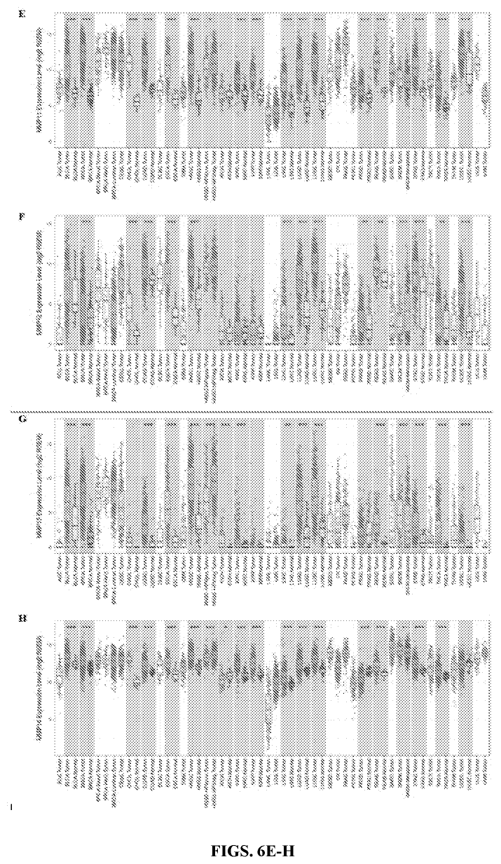

The inventors also analyzed human proteases expression level between tumor and adjacent normal tissues. The DiffExp module of TIMER (Tumor IMmune Estimation Resource) provides the comparison of gene expression levels for all samples from TCGA (The Cancer Genome Atlas). The assessed most of the tumor associated enzymes and found that MMP-1, MMP-3, MMP-9, MMP-10, MMP-11, MMP-12, MMP-13, and MMP-14 are these proteases which were significantly unregulated in most of tumors with relatively low background in normal tissues (FIGS. 6A-H). These proteases are excellent candidates for human IFN-Prodrug design to achieve maximum antitumor effect and minimum side effect on normal tissues. Interestingly, the consistent data of MMP-14 expression level in both mouse and human samples enhance the inventors' confidence in using MMP-14 substrate in IFN-Prodrug.

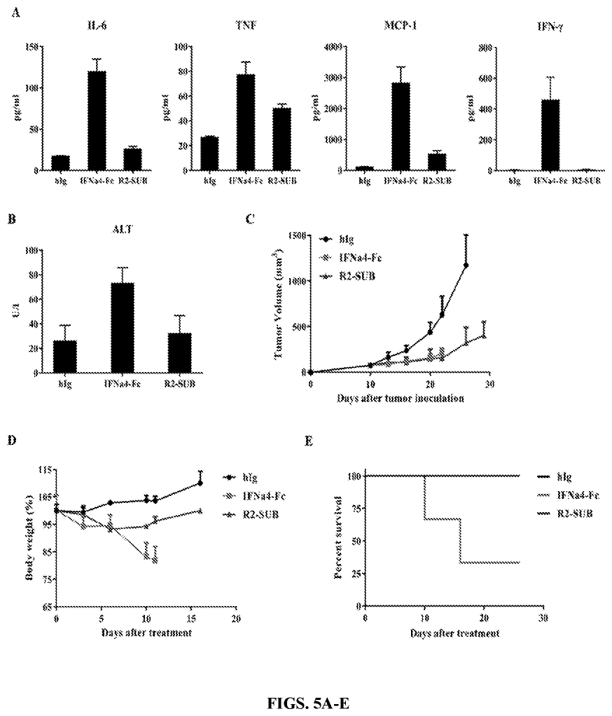

Therefore, the inventors engineered and purified the IFN-Prodrug based on the IFNAR2 and MMP-14 substrate (SGRSENIRTA)²⁵. They investigated the potential safety benefits and anti-tumor efficacy in vivo. Mice with established B16-OVA melanoma tumors were treated with R2-SUB, IFNa4-Fc, or hIg control, at a dosing of 1 mM with three injections for every three days. The inventors compared the relative safety of IFN-Prodrug and IFNa4-Fc. High levels of inflammation cytokines in serum, including IL-6, TNF, MCP-1, and IFN-g, were observed in IFNa4-Fc group on the day after the third treatment. In contrast, mice treated with R2-SUB showed very low levels of these inflammation cytokines (FIG. 5A). The ALT level, indicating liver toxicity, also showed similar results (FIG. 5B).

The inventors continued to monitor tumor growth and body weight. At a time point 12 days after treatment, R2-SUB had inhibited tumor growth by 76%, and IFNa4-Fc by 67% relative to the hIg-treated control group (FIG. 5C). However, at this time point, mice from IFNa4-Fc treated group were sick (hatched, inactive, and thin), likely due to the severe side effects of IFN. They next compared the body weight kinetics. The data showed that mice from IFNa4-Fc group started to lose weight quickly after the third treatment, however, the body weight of mice from R2-SUB group did not change significantly after three treatments (FIG. 5D). Finally, the inventors showed, in a survival curve based on toxicity, that the R2-SUB treated group had 100% survival, while the IFNa4-Fc treated group showed 33% survival (FIG. 5E).

Human IFN-Prodrug Based on Interferon Alpha and Beta Receptor (IFNAR).

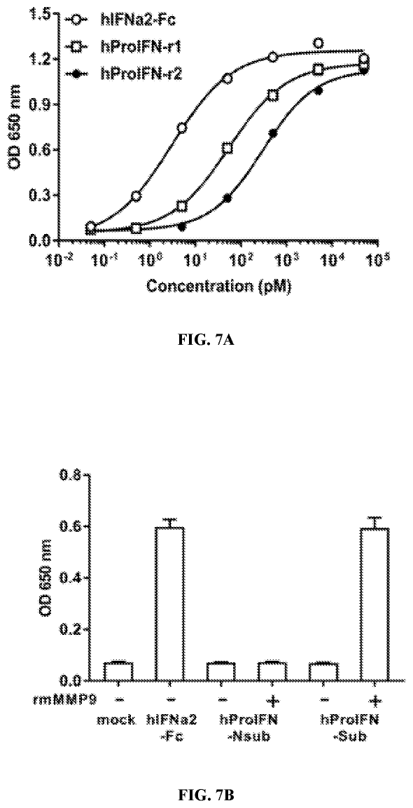

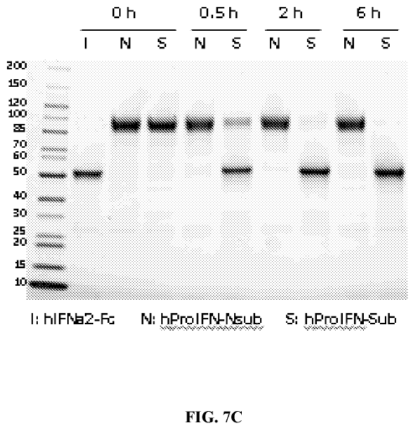

To determine whether the ECD of either human IFNAR1 or IFNAR2 is capable of blocking human interferon alpha 2 activity, the inventors purified human IFNa2-Fc, R1-IFNa2-Fc, and R2-IFNa2-Fc. The linker of R1-IFNa2-Fc or R2-IFNa2-Fc is 15-amino acid of triple Gly-Gly-Gly-Gly-Ser peptide, which is a flexible liker allowing for interaction between domains²³. They used 293T-Dual™ hSTING-R232 reporter cells which express the SEAP (secreted embryonic alkaline phosphatase) reporter gene under the control of the IFN-β minimal promoter fused to five NF-κB and AP-1 binding sites. 293T-Dual™ hSTING-R232 cells are responsive to human IFN-α and IFN-β. Human IFNa2-Fc, R1-IFNa2-Fc, and R2-IFNa2-Fc were serial-diluted in 10 fold from 50 nM for SEAP reporter assay. Compared to human IFNa2-Fc, R1-IFNa2-Fc showed decrease of IFN activity by 10-fold, while R2-IFNa2-Fc showed decrease of IFN activity by 100-fold (FIG. 7A). Thus, IFNAR2 is a better blocking reagent for human IFN.

To determine whether the activity of the activated human IFN-Prodrug was comparable to that of IFNa2-Fc, the inventors purified human R2-NSUB and R2-SUB. The linker of R2-SUB is a 16-amino acid peptide carrying the 6-residue protease-cleavable substrate (PVGLIG)²⁴, which can be cut by MMP-2 or MMP-9, flanked by flexible Gly-Gly-Gly-Gly-Ser peptides on both sides. The R2-NSUB without protease-cleavable substrate were used as control constructs. R2-NSUB or R2-SUB were incubated with rmMMP-9 at 37° C. for 0, 0.5, 2, or 6 hours. Human R2-SUB was cleaved by rmMMP-9 on a time-dependent manner. After 6 hours, the cleavage efficacy was 100% (FIG. 7C). Activated human R2-SUB showed comparable IFN activity relative to that of human IFNa2-Fc, while the activity of R2-NSUB did not change after enzyme inoculation (FIG. 7B).

Thus, the biological activity of protease-activated human IFN-Prodrug was fully restored in IFNAR2-based form to that the parental IFNa4-Fc. Moreover, the human IFNAR2-based IFN-Prodrug appeared remarkably reduced in its biological activity prior to protease cleavage, suggesting that the human IFN-Prodrug would remain safe in vivo until cleaved, and thereafter increasing in therapeutic activity at the local tumor microenvironment in human.

Thus, the IFN-Prodrug studied here makes the enhanced targeting of type I interferon to tumor tissue possible by reducing toxicity in normal tissues. The anti-tumor efficacy of IFN-Prodrug can be tailored based on the protease activity in each tumor, making the IFN-Prodrug a personalized drug with selected cleavable substrate linker in specific situations, giving the best result. Additionally, the IFN-Prodrug can be designed to carry multiple IFN domains using different linkers that are sensitive to different proteases, thereby significantly improving its therapeutic effect.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VII. REFERENCES

The following references, to the extent that they provide exemplary procedural or other details supplementary to those set forth herein, are specifically incorporated herein by reference.
Almendro et al., J. Immunol., 157(12):5411-5421, 1996.
Antonelli et al., Cytokine Growth Factor Rev 26, 121-131, 2015.
Ausubel et al., Current Protocols in Molecular Biology, Greene Publishing Associates and Wiley Interscience, N.Y., 1994.
Bates, Mol. Biotechnol., 2(2):135-145, 1994.
Battraw and Hall, Theor. App. Genet., 82(2):161-168, 1991.
Bellus, J. Macromol. Sci. Pure Appl. Chem., A31(1): 1355-1376, 1994.
Bhattacharjee et al., J Plant Bioch. Biotech., 6(2):69-73. 1997.
Burnette et al., Cancer Res 71, 2488-2496, 2011.
Capaldi et al., Biochem. Biophys. Res. Comm., 74(2):425-433, 1977.
Carbonelli et al., FEMS Microbiol. Lett., 177(1):75-82, 1999.
Chen and Okayama, Mol. Cell Biol., 7(8):2745-2752, 1987.
Christou et al., Proc. Natl. Acad. Sci. USA, 84(12):3962-3966, 1987.
Cocea, Biotechniques, 23(5):814-816, 1997.
D'Halluin et al., Plant Cell, 4(12):1495-1505, 1992.
De Jager et al., Semin. Nucl. Med., 23(2):165-179, 1993.
Desnoyers et al., Sci Transl Med 5, 207ra144, 2013.
Doolittle and Ben-Zeev, Methods Mol. Biol., 109, :215-237, 1999.
EPO 0273085
Erster, et al., J Control Release 161, 804-812, 2012.
Fechheimer et al., Proc Natl. Acad. Sci. USA, 84:8463-8467, 1987.
Ferrantini et al., Biochimie 89, 884-893, 2007.
Fraley et al., Proc. Natl. Acad. Sci. USA, 76:3348-3352, 1979.
Frohman, In: PCR Protocols: A Guide To Methods And Applications, Academic Press, N.Y., 1990.
Gabrilovich & Nagaraj, Nat Rev Immunol 9, 162-174, 2009.
GB Application No. 2 202 328
Ghosh and Bachhawat, In: Liver Diseases, Targeted Diagnosis and Therapy Using Specific Receptors and Ligands, Wu et al. (Eds.), Marcel Dekker, NY, 87-104, 1991.
Gopal, Mol. Cell Biol., 5:1188-1190, 1985.
Graham and Van Der Eb, Virology, 52:456-467, 1973.
Greene et al., Immunology Today, 10:272, 1989.
Harland and Weintraub, J. Cell Biol., 101(3):1094-1099, 1985.
He et al., Plant Cell Reports, 14 (2-3):192-196, 1994.
Hou and Lin, Plant Physiology, 111:166, 1996.
Imai et al., Nephrologie, 19(7):397-402, 1998.
Kaeppler et al., Plant Cell Rep., 8:415-418, 1990.
Kaneda et al., Science, 243:375-378, 1989.
Kato et al., J. Biol. Chem., 266:3361 3364, 1991.
Kato et al., N Engl J Med, 350:1713-21, 2004.
Khanna, Immunol Cell Biol 76, 20-26, 1998.
Kraus et al., FEBS Lett., 428(3):165-170, 1998.
Kreutzer et al., J Dtsch Dermatol Ges 2, 689-694, 2004.
Kwoh et al., Proc. Natl. Acad. Sci. USA, 86:1173, 1989.
Lareyre et al., J. Biol. Chem., 274(12):8282-8290, 1999.
Lazzeri, Methods Mol. Biol., 49:95-106, 1995.
Lee et al., Clin Cancer Res, 14:7397-404, 2008.
Lee et al., Environ. Mol. Mutagen., 13(1):54-59, 1989.
Levenson et al., Hum. Gene Ther 9(8):1233-1236, 1998.
Lotrich, Dialogues Clin Neurosci 11, 417-425, 2009.
Macejak and Sarnow, Nature, 353:90-94, 1991.
Moschos & Kirkwood, Cytokine Growth Factor Rev 18, 451-458, 2007.
Nakamura et al., In: Handbook of Experimental Immunology (4th Ed.), Weir et al. (Eds), 1:27, Blackwell Scientific Publ., Oxford, 1987.
Nicolau and Sene, Biochim. Biophys. Acta, 721:185-190, 1982.
Nicolau et al., Methods Enzymol., 149:157-176, 1987.
Nomoto et al., Gene, 236(2):259-271, 1999.
Ohara et al., Proc. Natl. Acad. Sci. USA, 86:5673-5677, 1989.
Olaussen et al., Curr Opin Pulm Med, 13:284-9, 2007.
Olaussen et al., N Engl J Med, 355:983-91, 2006.
Omirulleh et al., Plant Mol. Biol., 21(3):415-428, 1993.
Pace et al., J Immunol 184, 5969-5979, 2010.
Parker et al., Nat Rev Cancer 16, 131-144, 2016.
PCT Appln. WO 92/17598
PCT Appln. WO 94/09699
Pelletier and Sonenberg, Nature, 334(6180):320-325, 1988.
Perales et al., Proc. Natl. Acad. Sci. USA, 91:4086 4090, 1994.
Potrykus et al., Mol. Gen. Genet., 199(2):169-177, 1985.
Potter et al., Proc. Natl. Acad. Sci. USA, 81:7161-7165, 1984.
Remington's Pharmaceutical Sciences, 15th Ed., 33:624-652, 1990.
Rhodes et al., Methods Mol. Biol., 55:121 131, 1995.
Rippe, et al., Mol. Cell Biol., 10:689-695, 1990.
Roepman et al., Clin Cancer Res, 15:284-90, 2009.
Sambrook et al., In: Molecular Cloning: A Laboratory Manual, 2nd Ed., Cold Spring Harbor Laboratory Press, Cold Spring Harbor, N.Y., 1989.
Schiavoni et al., Cancer Res 71, 768-778, 2011.
Schmidt et al., Front Immunol 3, 51, 2012.
Sharma et al., Cell 168, 707-723, 2017.
Sleijfer et al., Pharm World Sci 27, 423-431, 2005.
Srivastava et al., J Exp Med 211, 961-974, 2014.
Stagg et al., Proc Nat'l Acad Sci USA 108, 7142-7147, 2011.
Stella et al., Drugs 29, 455-473, 1985.
Tsukada et al., Plant Cell Physiol., 30(4)599-604, 1989.
Tsumaki et al., J. Biol. Chem., 273(36):22861-22864, 1998.
Tur-Kaspa et al., Mol. Cell Biol., 6:716-718, 1986.
U.S. Pat. No. 3,817,837
U.S. Pat. No. 3,850,752
U.S. Pat. No. 3,939,350
U.S. Pat. No. 3,939,350
U.S. Pat. No. 3,996,345
U.S. Pat. No. 4,275,149
U.S. Pat. No. 4,277,437
U.S. Pat. No. 4,366,241
U.S. Pat. No. 4,415,723
U.S. Pat. No. 4,458,066
U.S. Pat. No. 4,683,195
U.S. Pat. No. 4,683,202
U.S. Pat. No. 4,684,611
U.S. Pat. No. 4,800,159
U.S. Pat. No. 4,879,236
U.S. Pat. No. 4,883,750
U.S. Pat. No. 4,952,500
U.S. Pat. No. 5,143,854
U.S. Pat. No. 5,202,231
U.S. Pat. No. 5,242,974
U.S. Pat. No. 5,279,721
U.S. Pat. No. 5,288,644
U.S. Pat. No. 5,302,523
U.S. Pat. No. 5,322,783
U.S. Pat. No. 5,324,633
U.S. Pat. No. 5,384,253
U.S. Pat. No. 5,384,261
U.S. Pat. No. 5,405,783
U.S. Pat. No. 5,412,087
U.S. Pat. No. 5,424,186
U.S. Pat. No. 5,429,807
U.S. Pat. No. 5,432,049
U.S. Pat. No. 5,436,327
U.S. Pat. No. 5,445,934
U.S. Pat. No. 5,464,765
U.S. Pat. No. 5,468,613
U.S. Pat. No. 5,470,710
U.S. Pat. No. 5,472,672
U.S. Pat. No. 5,492,806
U.S. Pat. No. 5,503,980
U.S. Pat. No. 5,510,270
U.S. Pat. No. 5,525,464
U.S. Pat. No. 5,527,681
U.S. Pat. No. 5,529,756
U.S. Pat. No. 5,532,128
U.S. Pat. No. 5,538,877
U.S. Pat. No. 5,538,880
U.S. Pat. No. 5,545,531
U.S. Pat. No. 5,547,839
U.S. Pat. No. 5,550,318
U.S. Pat. No. 5,554,501
U.S. Pat. No. 5,556,752
U.S. Pat. No. 5,561,071
U.S. Pat. No. 5,563,055
U.S. Pat. No. 5,571,639
U.S. Pat. No. 5,580,726
U.S. Pat. No. 5,580,732
U.S. Pat. No. 5,580,859
U.S. Pat. No. 5,589,466
U.S. Pat. No. 5,593,839
U.S. Pat. No. 5,599,672
U.S. Pat. No. 5,599,695
U.S. Pat. No. 5,610,042
U.S. Pat. No. 5,610,287
U.S. Pat. No. 5,624,711
U.S. Pat. No. 5,631,134
U.S. Pat. No. 5,639,603
U.S. Pat. No. 5,654,413
U.S. Pat. No. 5,656,610
U.S. Pat. No. 5,658,734
U.S. Pat. No. 5,661,028
U.S. Pat. No. 5,665,547
U.S. Pat. No. 5,667,972
U.S. Pat. No. 5,670,488
U.S. Pat. No. 5,695,940
U.S. Pat. No. 5,700,637
U.S. Pat. No. 5,702,932
U.S. Pat. No. 5,736,524
U.S. Pat. No. 5,739,018
U.S. Pat. No. 5,739,169
U.S. Pat. No. 5,744,305
U.S. Pat. No. 5,780,448
U.S. Pat. No. 5,789,215
U.S. Pat. No. 5,795,715
U.S. Pat. No. 5,800,992
U.S. Pat. No. 5,801,005
U.S. Pat. No. 5,807,522
U.S. Pat. No. 5,824,311
U.S. Pat. No. 5,830,645
U.S. Pat. No. 5,830,880
U.S. Pat. No. 5,837,196
U.S. Pat. No. 5,840,873
U.S. Pat. No. 5,843,640
U.S. Pat. No. 5,843,650
U.S. Pat. No. 5,843,651
U.S. Pat. No. 5,843,663
U.S. Pat. No. 5,846,708
U.S. Pat. No. 5,846,709
U.S. Pat. No. 5,846,717
U.S. Pat. No. 5,846,726
U.S. Pat. No. 5,846,729
U.S. Pat. No. 5,846,783
U.S. Pat. No. 5,846,945
U.S. Pat. No. 5,847,219
U.S. Pat. No. 5,849,481
U.S. Pat. No. 5,849,486
U.S. Pat. No. 5,849,487
U.S. Pat. No. 5,849,497
U.S. Pat. No. 5,849,546
U.S. Pat. No. 5,849,547
U.S. Pat. No. 5,851,772
U.S. Pat. No. 5,853,990
U.S. Pat. No. 5,853,992
U.S. Pat. No. 5,853,993
U.S. Pat. No. 5,856,092
U.S. Pat. No. 5,858,652
U.S. Pat. No. 5,861,244
U.S. Pat. No. 5,863,732
U.S. Pat. No. 5,863,753
U.S. Pat. No. 5,866,331
U.S. Pat. No. 5,866,366
U.S. Pat. No. 5,871,928
U.S. Pat. No. 5,871,986
U.S. Pat. No. 5,876,932
U.S. Pat. No. 5,882,864
U.S. Pat. No. 5,889,136
U.S. Pat. No. 5,900,481
U.S. Pat. No. 5,905,024
U.S. Pat. No. 5,910,407
U.S. Pat. No. 5,912,124
U.S. Pat. No. 5,912,145
U.S. Pat. No. 5,912,148
U.S. Pat. No. 5,916,776
U.S. Pat. No. 5,916,779
U.S. Pat. No. 5,919,626
U.S. Pat. No. 5,919,630
U.S. Pat. No. 5,922,574
U.S. Pat. No. 5,925,517
U.S. Pat. No. 5,925,565
U.S. Pat. No. 5,928,862
U.S. Pat. No. 5,928,869
U.S. Pat. No. 5,928,905
U.S. Pat. No. 5,928,906
U.S. Pat. No. 5,928,906
U.S. Pat. No. 5,929,227
U.S. Pat. No. 5,932,413
U.S. Pat. No. 5,932,451
U.S. Pat. No. 5,935,791
U.S. Pat. No. 5,935,819
U.S. Pat. No. 5,935,825
U.S. Pat. No. 5,939,291
U.S. Pat. No. 5,942,391
U.S. Pat. No. 5,945,100
U.S. Pat. No. 5,981,274
U.S. Pat. No. 5,994,624
U.S. Pat. No. 6,004,755
U.S. Pat. No. 6,087,102
U.S. Pat. No. 6,368,799
U.S. Pat. No. 6,383,749
U.S. Pat. No. 6,506,559
U.S. Pat. No. 6,573,099
U.S. Pat. No. 6,617,112
U.S. Pat. No. 6,638,717
U.S. Pat. No. 6,720,138
U.S. Patent Pubin. 2002/0168707
U.S. Patent Pubin. 2003/0051263
U.S. Patent Pubin. 2003/0055020
U.S. Patent Pubin. 2003/0159161
U.S. Patent Pubin. 2004/0064842
U.S. Patent Pubin. 2004/0265839
U.S. Patent Pubin. 2008/0009439
Wagner et al., Proc. Natl. Acad. Sci. USA 87(9):3410-3414, 1990.
Walker et al., Nucleic Acids Res. 20(7):1691-1696, 1992.
Weir et al., Nature, 450:893-8, 2007.
Woo et al., Immunity 41, 830-842, 2014.
Wu and Wu, Adv. Drug Delivery Rev., 12:159 167, 1993.
Wu and Wu, J. Biol. Chem., 262:4429 4432, 1987.
Wu et al., Biochem. Biophys. Res. Commun., 233(1):221-226, 1997.
Wu et al., Cancer Res., 58(8): 1605-8, 1998.
Zhao-Emonet et al., Biochim. Biophys. Acta, 1442(2-3):109-119, 1998.
Zhou et al., Nature, 361(6412):543-547, 1993.
Zitvogel et al., Nat Rev Immunol 15, 405-414, 2015.
Zoglmeier et al., Clin Cancer Res 17, 1765-1775, 2011.

1. An interferon prodrug comprising:
(a) an interferon alpha and beta receptor (IFNAR) domain that retains IFN binding activity;
(b) a type 1 interferon (IFN) domain that retains type 1 interferon activity when not engaged by said IFNAR domain;
(c) an immunoglobulin (Ig) Fc domain,
(d) a first linker fused at one end to the N-terminus of said IFN and fused at the other end to said IFNAR, wherein said first linker is protease cleavable; and
(e) a second linker fused at one end to the C-terminus of said IFN and fused at the other end to the N-terminus of said Ig Fc domain.

2. The fusion protein of claim 1, wherein said Ig is IgG, such as IgG1 or IgG2.

3. The fusion protein of claim 1, wherein said interferon prodrug contains two copies of said type 1 IFN domain.

4. The fusion protein of claim 1, wherein said interferon prodrug contains more than two copies of said type 1 IFN domain.

5. The fusion protein of claim 1, wherein said first linker is cleavable by one or more matrix metalloproteinases, such as MMP1, MMP3, MMP9, MMP10, MMP11, MMP12, MMP13 or MMP14.

6. The fusion protein of claim 1, wherein said first linker is cleavable by UPA, FAPa and/or Cathepsin B.

7. The fusion protein of claim 1, wherein said linker is G4S-SUB1-G4S-SUB2-G4S-SUB3-G4S, wherein SUB1-3 are distinct enzyme cleavage sites.

8. The fusion protein of claim 1, wherein said IFNAR is IFNAR1.

9. The fusion protein of claim 1, wherein said IFNAR is IFNAR2.

10. The fusion protein of claim 1, wherein said IFN is IFN-α, IFN-β, IFN-κ, IFN-δ, IFN-ε, IFN-τ, IFN-ω or IFN-ξ.

11. A nucleic acid construct encoding an interferon prodrug comprising:
(a) an interferon alpha and beta receptor (IFNAR) domain that retains IFN binding activity;
(b) a type 1 interferon (IFN) domain that retains type 1 interferon activity when not engaged by said IFNAR domain;
(c) an immunoglobulin (Ig) Fc domain,
(d) a first linker fused at one end to the N-terminus of said IFN and fused at the other end to said IFNAR, wherein said first linker is protease cleavable;
(e) a second linker fused at one end to the C-terminus of said IFN and fused at the other end to the N-terminus of said Ig Fc domain; and
(f) a promoter positioned 5′ to the 5′ end of said IFNα domain.

12. The nucleic acid construct of claim 11, wherein said Ig is IgG, such as IgG1 or IgG2.

13. The nucleic acid construct of claim 11, wherein said interferon prodrug contains two copies of said type 1 IFN domain.

14. The nucleic acid construct of claim 11, wherein said interferon prodrug contains more than two copies of said type 1 IFN domain.

15. The nucleic acid construct of claim 11, wherein said first linker is cleavable by a matrix metalloproteinase, such as MMP1, MMP3, MMP9, MMP10, MMP11, MMP12, MMP13 and/or MMP14.

16. The nucleic acid construct claim 11, wherein said first linker is cleavable by UPA, FAPa and/or Cathepsin B.

17. The nucleic acid construct of claim 11, wherein said linker is G4S-SUB1-G4S-SUB2-G4S-SUB3-G4S, wherein SUB1-3 are distinct enzyme cleavage sites.

18. The nucleic acid construct claim 11, wherein said IFNAR is IFNAR1.

19. The nucleic acid construct claim 11, wherein said IFNAR is IFNAR2.

20. The nucleic acid construct of claim 11, wherein said IFN is IFN-α, IFN-β, IFN-κ, IFN-δ, IFN-ε, IFN-τ, IFN-ω or IFN-ξ.

21. A recombinant cell expressing an interferon prodrug according to claim 1.

22. A recombinant cell comprising a nucleic acid construct according to claim 11.

23. A method of expressing an interferon prodrug comprising culturing the cell of claim 21.

24. A method of expressing an interferon prodrug comprising culturing the cell of claim 22.

25. (canceled)

26. A method of treating cancer comprising administering to a subject in need thereof an interferon prodrug according to claim 1.

27. The method of claim 26, further comprising the step of assessing protease expression in a cancer cell obtained from said subject.

28. The method of claim 27, wherein said cancer cell is obtained from a biopsy.

29. The method of claim 27, wherein said cancer cell is a circulating tumor cell.

30. The method of claim 26, wherein said cancer is lung cancer, breast cancer, brain cancer, oral cancer, esophageal cancer, head & neck cancer, skin cancer, stomach cancer, liver cancer, pancreatic cancer, renal cancer, ovarian cancer, prostate cancer, bladder cancer, colon cancer, testicular cancer, uterine cancer, cervical cancer, lymphoma, or leukemia.

31. The method of claim 26, wherein said cancer is primary, recurrent, metastatic or multi-drug resistant.

32. The method of claim 26, wherein said patient has previously received surgical therapy, chemotherapy, radiotherapy, hormonal therapy or immunotherapy.

33. The method of claim 26, further comprising treating said subject with a second cancer therapy.

34. The method of claim 33, wherein said second cancer therapy is surgical therapy, chemotherapy, radiotherapy, hormonal therapy or immunotherapy.

35. The method of claim 26, wherein said subject is a human or a non-human mammal.

36. The method of claim 26, further comprising administering said interferon prodrug more than once.

37. The method of claim 36, wherein said interferon prodrug is administered daily, every other day, weekly, every other week, or monthly.

38. The method of claim 26, wherein said interferon prodrug is administered systemically.

39. The method of claim 26, wherein said interferon prodrug is administered intratumorally, local to a tumor or regional to a tumor.

40. The method of claim 26, wherein treating comprises one or more of slowing tumor growth, halting tumor growth, reduction in tumor size or burden, increasing survival as compared to an untreated subject, inducing cancer remission, inducing tumor cell apoptosis, or inducing tumor necrosis.


SEQUENCE LISTING (13 sequences)

SEQ ID NO: 1 - protein, 429 aa, Mus musculus
MLAVVGAAALVLVAGAPWVLPSAAGGENLKPPENIDVYIIDDNYTLKWSSHGESMGSVTFSAEYRTKDEAKWLKVPECQHTTTTKCEFSLLDTNVYIKTQFRVRAEEGNSTSSWNEVDPFIPFYTAHMSPPEVRLEAEDKAILVHISPPGQDGNMWALEKPSFSYTIRIWQKSSSDKKTINSTYYVEKIPELLPETTYCLEVKAIHPSLKKHSNYSTVQCISTTVANKMPVPGNLQVDAQGKSYVLKWDYIASADVLFRAQWLPGYSKSSSGSRSDKWKPIPTCANVQTTHCVFSQDTVYTGTFFLHVQASEGNHTSFWSEEKFIDSQKHILPPPPVITVTAMSDTLLVYVNCQDSTCDGLNYEIIFWENTSNTKISMEKDGPEFTLKNLQPLTVYCVQARVLFRALLNKTSNFSEKLCEKTRPGSFST

SEQ ID NO: 2 - protein, 242 aa, Mus musculus
MRSRCTVSAVGLLSLCLVVSASLETITPSAFDGYPDEPCTINITIRNSRLILSWELENKSGPPANYTLWYTVMSKDENLTKVKNCSDTTKSSCDVTDKWLEGMESYVVAIVIVHRGDLTVCRCSDYIVPANAPLEPPEFEIVGFTDHINVTMEFPPVTSKIIQEKMKTTPFVIKEQIGDSVRKKHEPKVNNVTGNFTFVLRDLLPKTNYCVSLYFDDDPAIKSPLKCIVLQPGQESGLSESA

SEQ ID NO: 3 - protein, 436 aa, Homo sapiens
MMVVLLGATTLVLVAVAPWVLSAAAGGKNLKSPQKVEVDIIDDNFILRWNRSDESVGNVTFSFDYQKTGMDNWIKLSGCQNITSTKCNFSSLKLNVYEEIKLRIRAEKENTSSWYEVDSFTPFRKAQIGPPEVHLEAEDKAIVIHISPGTKDSVMWALDGLSFTYSLVIWKNSSGVEERIENIYSRHKIYKLSPETTYCLKVKAALLTSWKIGVYSPVHCIKTTVENELPPPENIEVSVQNQNYVLKWDYTYANMTFQVQWLHAFLKRNPGNHLYKWKQIPDCENVKTTQCVFPQNVFQKGIYLLRVQASDGNNTSFWSEEIKFDTEIQAFLLPPVFNIRSLSDSFHIYIGAPKQSGNTPVIQDYPLIYEIIFWENTSNAERKIIEKKTDVTVPNLKPLTVYCVKARAHTMDEKLNKSSVFSDAVCEKTKPGNTSK

SEQ ID NO: 4 - protein, 243 aa, Homo sapiens
MLLSQNAFIFRSLNLVLMVYISLVFGISYDSPDYTDESCTFKISLRNFRSILSWELKNHSIVPTHYTLLYTIMSKPEDLKVVKNCANTTRSFCDLTDEWRSTHEAYVTVLEGFSGNTTLFSCSHNFWLAIDMSFEPPEFEIVGFTNHINVMVKFPSIVEEELQFDLSLVIEEQSEGIVKKHKPEIKGNMSGNFTYIIDKLIPNTNYCVSVYLEHSDEQAVIKSPLKCTLLPPGQESESAESAK

SEQ ID NO: 5 - protein, 6 aa, Artificial sequence
  Synthetic peptide
PVGLIG

SEQ ID NO: 6 - protein, 10 aa, Artificial sequence
  Synthetic pedptide
GGYAELRMGG

SEQ ID NO: 7 - protein, 8 aa, Artificial sequence
  Synthetic pedptide
GPRPFNYL

SEQ ID NO: 8 - protein, 8 aa, Artificial sequence
  Synthetic pedptide
GGALGLSL

SEQ ID NO: 9 - protein, 8 aa, Artificial sequence
  Synthetic pedptide
GPMSYNAL

SEQ ID NO: 10 - protein, 10 aa, Artificial sequence
  Synthetic pedptide
SGRSENIRTA

SEQ ID NO: 11 - protein, 9 aa, Artificial sequence
  Synthetic pedptide
TSGPNQEQK

SEQ ID NO: 12 - protein, 4 aa, Artificial sequence
  Synthetic pedptide
GFLG

SEQ ID NO: 13 - protein, 5 aa, Artificial sequence
  Synthetic pedptide
GGGGS